(19) United States
(12) Patent Application Publication
(10) Pub. No.: US 2024/0358896 A1
(43) Pub. Date: Oct. 31, 2024

(54) SUSPENSIONS OF RADIOPAQUE CROSSLINKED HYDROGEL PARTICLES IN CARRIER FLUIDS CONTAINING BIOCOMPATIBLE HYDROPHILIC POLYMERS

(71) Applicant: Boston Scientific Scimed, Inc., Maple Grove, MN (US)

(72) Inventors: Yen-Hao Hsu, Shrewsbury, MA (US); Peijiang Wang, Brighton, MA (US); Cristian Parisi, Boston, MA (US); Drew Carlton Wolos, Allston, MA (US)

(73) Assignee: Boston Scientific Scimed, Inc., Maple Grove, MN (US)

(21) Appl. No.: 18/649,602

(22) Filed: Apr. 29, 2024

Related U.S. Application Data
(60) Provisional application No. 63/462,852, filed on Apr. 28, 2023.

(51) Int. Cl. A61L 27/54; A61L 27/26; A61L 27/52; A61L 27/58

(52) U.S. Cl. CPC A61L 27/54; A61L 27/26; A61L 27/52; A61L 27/58; A61L 2400/06

(57) ABSTRACT

In some embodiments, the present disclosure pertains to an injectable suspension that contains radiopaque crosslinked hydrogel particles in a carrier fluid that comprises one or more types of linear hydrophilic polymers. In some embodiments, the present disclosure pertains to kits that comprise a syringe preloaded with an injectable suspension that contains radiopaque crosslinked hydrogel particles in a carrier fluid that comprises one or more types of linear hydrophilic polymers. In some embodiments, the present disclosure pertains to methods of treatment comprising injecting into a subject an injectable suspension that contains radiopaque crosslinked hydrogel particles in a carrier fluid that comprises one or more types of linear hydrophilic polymers.

### CROSS-REFERENCE TO RELATED APPLICATIONS

This application claims the benefit of U.S. Provisional Patent Application Ser. No. 63/462,852 filed on Apr. 28, 2023, the disclosure of which is incorporated herein by reference.

### FIELD

The present disclosure relates to crosslinked hydrogel particles and to carrier fluids containing biocompatible hydrophilic polymers that are useful for forming suspensions of crosslinked hydrogel particles. The suspensions of crosslinked hydrogel particles are useful, for example, in various medical applications.

### BACKGROUND

Bioresorbable hydrogels with rapid crosslinking reaction rate in vivo, known by the trade name of SpaceOAR®, have become a prominent biomaterial and obtained clinical success in creating the space between prostate and rectum, tremendously improving patient safety during the cancer therapies. A further improvement based on this application is that some of 8-Arm PEG branches are functionalized with 2,3,5-triiiodobenzamide (TIB) groups, replacing part of the activated ester end groups, succinimidyl glutarate (SG), in order to provide intrinsic radiopacity to the hydrogels themselves for CT-visibility. This hydrogel, known by the trade name of SpaceOAR Vue®, is the next generation of SpaceOAR® for prostate medical applications.

While the above approach is effectual, some issues arise as a result of the incorporation of the TIB functional group. First, in order to functionalize TIB on 8-arm PEG, one succinimidyl glutarate (SG) binding site is sacrificed for each functionalized arm, reducing capacity and efficiency of the crosslinking reaction in vivo. Moreover, SG groups can start their degradation in acidic pH environments, which could potentially cause longer gel times and faster dissipation of crosslinked hydrogels in vivo. Furthermore, the functionalized 8-arm PEG is co-injected with trilysine as a crosslinking agent, limiting the ability to pause during injection and requiring a double-barrel syringe.

For these and other reasons, alternative strategies for forming radiopaque injectable hydrogels are desired.

### SUMMARY

The present disclosure provides an alternative approach to that described above.

In some embodiments, the present disclosure pertains to an injectable suspension that contains radiopaque crosslinked hydrogel particles in a carrier fluid that comprises one or more types of linear hydrophilic polymers.

In some embodiments, the linear hydrophilic polymer is selected from a poly-2-oxazoline, a polyvinylpyrrolidone, a polyacrylamide, a polysaccharide, a polypeptide, a polyvinyl alcohol, and a poly(2-hydroxyethyl methacrylate).

In some embodiments, which can be used in conjunction the above embodiments, the linear hydrophilic polymer has a weight average molecular weight (M_{w}) ranging from 1 kDa to 60 kDa.

In some embodiments, which can be used in conjunction with the above embodiments, the carrier fluid contains between 1 wt % and 35 wt % of the one or more types of linear hydrophilic polymers.

In some embodiments, which can be used in conjunction with the above embodiments, the radiopaque crosslinked hydrogel particles comprise one or more radiopaque atoms selected from Br, I, Bi, Ba, Gd, Ta, Zn, W and Au.

In some embodiments, which can be used in conjunction with the above embodiments, the radiopaque crosslinked hydrogel particles comprise covalently attached iodine atoms.

In some embodiments, which can be used in conjunction with the above embodiments, the radiopaque crosslinked hydrogel particles comprise a crosslinked polymer that comprises one or more of the following: a poly(alkylene oxide) chain, a poly(N-vinyl pyrrolidone) chain, a poly(2-oxazoline) chain, a polyacrylamide chain, a polysaccharide chain, a polypeptide chain, a polyvinyl alcohol chain, and a poly(2-hydroxyethyl methacrylate) chain.

In some embodiments, which can be used in conjunction with the above embodiments, the radiopaque crosslinked hydrogel particles comprise a crosslinked polymer having a core region and a plurality of polymer arms linked to the core region, the polymer arms each comprising a hydrophilic polymer chain.

In some embodiments, which can be used in conjunction with the above embodiments, a portion of the polymer arms comprise a radiopaque-atom-containing moiety at the end.

In some embodiments, which can be used in conjunction with the above embodiments, the radiopaque crosslinked hydrogel particles comprise a crosslinked reaction product of (a) a reactive multi-arm polymer containing a plurality of first reactive moieties that comprise an electrophilic group and (b) a reactive compound having a plurality of second reactive moieties that comprise nucleophilic groups.

In some embodiments, which can be used in conjunction with the above embodiments, the injectable suspension contains between 1 wt % and 25 wt % (dry weight) radiopaque crosslinked hydrogel particles relative to the total weight of the suspension.

In some embodiments, which can be used in conjunction with the above embodiments, the radiopaque crosslinked hydrogel particles range 10 to 1500 microns in in longest dimension.

In some embodiments, which can be used in conjunction with the above embodiments, the carrier fluid contains an acidic buffer and has a pH ranging from 3 to 6.5.

In some embodiments, which can be used in conjunction with the above embodiments, the carrier fluid further comprises a therapeutic agent.

In some embodiments, which can be used in conjunction with the above embodiments, the injectable suspension has a radiopacity that is greater than 100 Hounsfield units (HU).

In some embodiments, an injectable suspension in accordance with any of the above embodiments is preloaded in a syringe.

In some embodiments, the present disclosure pertains to kits that comprise a syringe preloaded with an injectable suspension in accordance with any of the above embodiments and a delivery device.

In some embodiments, the delivery device comprises a catheter and/or a needle.

In some embodiments, the present disclosure pertains to methods of treatment comprising injecting an injectable suspension in accordance with any of the above embodiments into a subject.

In some embodiments, the hydrogel particle suspension is injected to provide spacing between tissues, injected to provide fiducial markers, injected for tissue augmentation or regeneration, injected as a filler or replacement for soft tissue, injected to provide mechanical support for compromised tissue, injected as a scaffold, and/or injected as a carrier for one or more therapeutic agents.

Potential benefits associated with the present disclosure include one or more of the following: physicians can pause injection without any risk of needle clogging during product injection and a single syringe is used. Moreover, radiocontrast and in vivo persistence are obtained.

The above and other aspects, embodiments, features and benefits of the present disclosure will be readily apparent from the following detailed description.

### BRIEF DESCRIPTION OF THE DRAWINGS

FIG. 1 schematically illustrates a catheter and a syringe that is loaded with a hydrogel particle suspension, in accordance with an embodiment of the present disclosure.

### DETAILED DESCRIPTION

The present disclosure pertains to suspensions of radiopaque crosslinked hydrogel particles in carrier fluids. The carrier fluid contains one or more types of linear hydrophilic polymers that act as a lubricant and significantly improve the injectability of the radiopaque crosslinked hydrogel particles by reducing injection force.

Linear hydrophilic polymers in accordance with the present disclosure can be selected from any of a variety of synthetic, natural, or hybrid synthetic-natural hydrophilic polymers. Examples of linear hydrophilic polymers include linear homopolymers and linear copolymers formed from one or more of the following hydrophilic monomers: ethylene oxide, propylene oxide, N-vinyl pyrrolidone, oxazoline monomers (e.g., 2-alkyl-2-oxazolines, for instance, 2-(C₁-C₆alkyl)-2-oxazolines, including various isomers, such as 2-methyl-2-oxazoline, 2-ethyl-2-oxazoline, 2-propyl-2-oxazoline, 2-isopropyl-2-oxazoline, 2-n-butyl-2-oxazoline, 2-n-hexyl-2-oxazoline, etc., and 2-phenyl-2-oxazoline), vinyl alcohol, allyl alcohol, hydroxyethyl acrylate, hydroxyethyl methacrylate, acrylamide, N-isopropylacrylamide, and amino acids.

Linear hydrophilic polymers may be selected, for example, from one or combination of any of the following homopolymers and copolymers: polyethers including poly(alkylene oxides) such as poly(ethylene oxide) (PEO) (also referred to as polyethylene glycol or PEG), poly(propylene oxide), poly(ethylene oxide-co-propylene oxide), poly(N-vinyl pyrrolidone), poly-2-oxazolines including poly(2-C₁-C₆-alkyl-2-oxazoline)s such as poly(2-methyl-2-oxazoline), poly(2-ethyl-2-oxazoline), poly(2-propyl-2-oxazoline), poly(2-isopropyl-2-oxazoline), and poly(2-n-butyl-2-oxazoline), poly (2-phenyl-2-oxazoline), poly(vinyl alcohol), poly(allyl alcohol), polyhydroxyethyl acrylate, polyhydroxyethyl methacrylate, polyacrylamide and derivatives of the same, such as poly(N-isopropylacrylamide), and polypeptides.

In some embodiments, the linear hydrophilic polymer is PEG. However, some patients have PEG allergies. Therefore, in other embodiments, non-PEG hydrophilic polymers are employed. In particular beneficial embodiments, the linear hydrophilic polymer may be selected from poly-2-oxazolines, with various side groups tuning the hydrophilicity of the same, polyvinylpyrrolidone, polyacrylamide, poly(N-isopropylacrylamide) or polypeptides.

Linear hydrophilic polymers in accordance with the present disclosure may vary in length but typically have a weight average molecular weight (M_{w}) ranging from 1 kDa to 60 kDa, for example, ranging anywhere from 1 kDa to 2 kDa to 5 kDa to 10 kDa to 15 kDa to 20 kDa to 30 kDa to 40 kDa to 50 kDa to 60 kDa (i.e., between any two of the preceding values), among other possible values.

The concentration of the one or more types of linear hydrophilic polymers in the carrier fluid may vary but typically contains between 1 wt % and 35 wt % relative to the weight of the carrier fluid, for example, ranging anywhere from 1 wt % to 2.5 wt % to 5 wt % to 10 wt % to.15 wt % to 20 wt % to 25 wt % to 30 wt % to 35 wt %.

In some embodiments, the carrier fluid contains a buffer that can, for example, stabilize the particles, maintain gel longevity and/or reduce required injection force.

For example, in some embodiments, the carrier fluid includes an acidic buffer and has a pH ranging from 3 to 6.5, for example, ranging from 3.5 to 4.5. Any buffer that is biocompatible and suitable for injection may be employed, with monobasic sodium phosphate one particular example.

The carrier fluid may further contain water, which may be, for example, in the form of water for injection, saline or phosphate buffered saline.

The concentration of water in the carrier fluid may vary but is typically at least 65 wt % relative to the weight of the carrier fluid.

The concentration of the radiopaque crosslinked hydrogel particles in the suspension may vary, but the suspension typically contains between 1 wt % and 25 wt % (dry weight) radiopaque crosslinked hydrogel particles relative to the total weight of the suspension (e.g., ranging anywhere from 1 wt % to 2.5 wt % to 5 wt % to 10 wt % to 15 wt % to 20 wt % to 25 wt %).

The radiopaque crosslinked hydrogel particles may vary in size and typically range between 10 and 1500 μm in longest dimension (e.g., diameter for a spherical particle, length for an elongate or rod-shaped particle, greatest width for a plate-like particle, etc.) (e.g., ranging from 10 μm to 25 μm to 50 μm to 100 μm to 250 μm to 500 μm to 1000 μm to 1500 μm).

In some embodiments, the suspension may further contain additional agents, examples of which are discussed further below.

The radiopaque crosslinked hydrogel particles of the present disclosure include crosslinked reaction products of (a) a reactive multi-arm polymer containing a plurality of first reactive moieties that comprise an electrophilic group and (b) a reactive compound having a plurality of second reactive moieties that comprise nucleophilic groups.

Reactive multi-arm polymers for forming the radiopaque crosslinked hydrogel particles include polymers that comprise a plurality of polymer arms linked to a core region. A portion or all of the polymer arms of the reactive multi-arm polymers comprise a hydrophilic polymer chain linked to the core region and a reactive moiety at the end of the polymer arm. Reactive multi-arm polymers include polymers having three, four, five, six, seven, eight, nine, ten, fifteen, twenty, or more arms.

In some embodiments, the reactive multi-arm polymers further comprises one or more radiopaque atoms, which may be selected, for example, from Br, I, Bi, Ba, Gd, Ta, Zn, W and Au. The one or more radiopaque atoms may be provided in the core of the reactive multi-arm polymer, between the core and at least one of the hydrophilic polymer chains, within at least one of the hydrophilic polymer chains, between at least one of the hydrophilic polymer chains and the reactive moiety at the end of the polymer arm, or at the end of at least one of the polymer arms. In certain cases, a first portion of the polymer arms will each contain a radiopaque-atom-containing moiety at the end of the polymer arm and the remainder of the polymer arms will each contain a reactive moiety at the end of the polymer arm.

In some embodiments, the first reactive moieties that comprise an electrophilic group may be selected from succinimide ester groups, imidazole ester groups, imidazole carboxylate groups and benzotriazole ester groups, among other possibilities. In some embodiments, the reactive moiety may be covalently linked to the polymer arms through a hydrolysable ester group. For instance, the reactive moiety may comprise a diester. In particular examples, the diester may be selected from a malonic-acid-based diester, a succinic-acid-based diester, a glutaric-acid-based diester and an adipic-acid-based diester. In certain embodiments, a succinimide group is linked to one ester of the diester and the other ester of the diester is linked to a hydrophilic polymer chain. Particular examples of such reactive moieties include succinimidyl malonate moieties, succinimidyl succinate moieties, succinimidyl glutarate moieties, and succinimidyl adipate moieties.

Hydrophilic polymer chains can be selected from any of a variety of synthetic, natural, or hybrid synthetic-natural hydrophilic polymer chains. Examples of hydrophilic polymer chains include homopolymer and copolymer chains formed from one or more of the following hydrophilic monomers: ethylene oxide, propylene oxide, N-vinyl pyrrolidone, oxazoline monomers (e.g., 2-alkyl-2-oxazolines, for instance, 2-(C₁-C₆alkyl)-2-oxazolines, including various isomers, such as 2-methyl-2-oxazoline, 2-ethyl-2-oxazoline, 2-propyl-2-oxazoline, 2-isopropyl-2-oxazoline, 2-n-butyl-2-oxazoline, 2-n-hexyl-2-oxazoline, etc., and 2-phenyl-2-oxazoline), vinyl alcohol, allyl alcohol, hydroxyethyl acrylate, hydroxyethyl methacrylate, N-isopropylacrylamide, amino acids and sugars.

Hydrophilic polymer chains may be selected, for example, from the following copolymer chains: polyether chains including poly(alkylene oxide) chains such as poly(ethylene oxide) (PEO) (also referred to as polyethylene glycol or PEG) chains, poly(propylene oxide) chains, poly(ethylene oxide-co-propylene oxide) chains, poly(N-vinyl pyrrolidone) chains, polyoxazoline chains including poly(2-C₁-C₆-alkyl-2-oxazoline chains) such as poly(2-methyl-2-oxazoline) chains, poly(2-ethyl-2-oxazoline) chains, poly(2-propyl-2-oxazoline) chains, poly(2-isopropyl-2-oxazoline) chains, and poly(2-n-butyl-2-oxazoline) chains, poly(2-phenyl-2-oxazoline) chains, poly(vinyl alcohol) chains, poly(allyl alcohol) chains, polyhydroxyethyl acrylate chains, polyhydroxyethyl methacrylate chains, and poly(N-isopropylacrylamide) chains.

Hydrophilic polymer chains for use in the multi-arm polymers of the present disclosure typically contain between 10 and 2000 monomer units (e.g., ranging from 10 to 20 to 50 to 100 to 200 to 500 to 1000 to 2000 monomer units). In some embodiments, the hydrophilic polymer chains have the same monomer composition as the linear hydrophilic polymers.

Core regions can be selected from a residue of a polyol comprising two or more hydroxyl groups, which is used to form the copolymer arms. In certain beneficial embodiments, the core region comprises a residue of a polyol that contains two, three, four, five, six, seven, eight, nine, ten, eleven, twelve or more hydroxyl groups.

Illustrative polyols may be selected, for example, from straight-chained, branched and cyclic aliphatic polyols including straight-chained, branched and cyclic polyhydroxyalkanes, straight-chained, branched and cyclic polyhydroxy ethers, including polyhydroxy polyethers, straight-chained, branched and cyclic polyhydroxyalkyl ethers, including polyhydroxyalkyl polyethers, straight-chained, branched and cyclic sugars and sugar alcohols, such as glycerol, mannitol, sorbitol, inositol, xylitol, quebrachitol, threitol, arabitol, erythritol, pentaerythritol, dipentaerythritol, tripentaerythritol, adonitol, hexaglycerol, dulcitol, fucose, ribose, arabinose, xylose, lyxose, rhamnose, galactose, glucose, fructose, sorbose, mannose, pyranose, altrose, talose, tagatose, pyranosides, sucrose, lactose, and maltose, polymers (defined herein as two or more units) of straight-chained, branched and cyclic sugars and sugar alcohols, including oligomers (defined herein as ranging from two to ten units, including dimers, trimers, tetramers, pentamers, hexamers, heptamers, octamers, enneamers and decamers) of straight-chained, branched and cyclic sugars and sugar alcohols, including the preceding sugars and sugar alcohols, starches, amylose, dextrins, cyclodextrins, as well as polyhydroxy crown ethers, and polyhydroxyalkyl crown ethers. Illustrative polyols also include aromatic polyols including 1,1,1-tris(4′-hydroxyphenyl) alkanes, such as 1,1,1-tris(4-hydroxyphenyl)ethane, and 2,6-bis(hydroxyalkyl)cresols, among others. Illustrative polyols also include polyhydroxylated polymers. For example, in some embodiments, the core region comprises a polyhydroxylated polymer residue such as a poly(vinyl alcohol) residue, poly(allyl alcohol), polyhydroxyethyl acrylate residue, or a polyhydroxyethyl methacrylate residue, among others. Such polyhydroxylated polymer residues may range, for example, from 5 to 1000 units in length, typically from 10 to 25 units in length.

In other embodiments, the core region comprises a silsesquioxane. A silsesquioxane is a compound that has a cage-like silicon-oxygen core that is made up of Si—O—Si linkages and tetrahedral Si vertices.—H groups or exterior organic groups may be covalently attached to the cage-like silicon-oxygen core. In the present disclosure, the organic groups comprise copolymer arms. Silsesquioxanes for use in the present disclosure include silsesquioxanes with 6 Si vertices, silsesquioxanes with 8 Si vertices, silsesquioxanes with 10 Si vertices, and silsesquioxanes with 12 Si vertices, which can act, respectively, as cores for 6-arm, 8-arm, 10-arm and 12-arm polymers. The silicon-oxygen cores are sometimes referred to as T6, T8, T10, and T12 cage-like silicon-oxygen cores, respectively (where T=the number of tetrahedral Si vertices). In all cases each Si atom is bonded to three O atoms, which in turn connect to other Si atoms. Silsesquioxanes include compounds of the chemical formula [RSiO_{3/2}]ₙ, where n is an integer of at least 6, commonly 6, 8, 10 or 12 (thereby having T₆, T₈, T₁₀ or T₁₂ cage-like silicon-oxygen core, respectively), and where R may be selected from an array of organic functional groups such as alkyl groups, aryl groups, alkoxyl groups, and polymeric arms, among others. The T₈ cage-like silicon-oxygen cores are widely studied and have the formula [RSiO_{3/2}]₈, or equivalently R₈Si₈O₁₂. Such a structure is shown here:

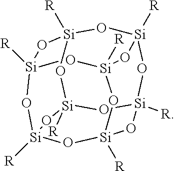

In the present disclosure, the R groups comprise the polymer arms described herein.

As previously noted, the radiopaque crosslinked hydrogel particles of the present disclosure include crosslinked reaction products of (a) a reactive multi-arm polymer containing a plurality of first reactive moieties that comprise an electrophilic group, such as those described above, and (b) a reactive compound having a plurality of second reactive moieties that comprise nucleophilic groups (e.g. amine-containing moieties and/or thiol-containing moieties, among others), wherein at least one of the reactive multi-arm polymer and the reactive compound having a plurality of second reactive moieties that comprise nucleophilic groups comprises one or more radiopaque atoms.

In various embodiments, the reactive compound having a plurality of second reactive moieties that comprise nucleophilic groups is a polyamino compound. In general, polyamino compounds suitable for use in the present disclosure include, for example, small molecule polyamines (e.g., containing at least two amine groups, for instance, from 3 to 20 amine groups or more in certain embodiments), polymers having amine side groups, and branched polymers having amine end groups, including dendritic polymers having amine end groups. Polyamino compounds suitable for use in the present disclosure include those that comprises a plurality of—(CH₂)ₓ-NH₂ groups where x is 0, 1, 2, 3, 4, 5 or 6. Polyamino compounds suitable for use in the present disclosure include polyamino compounds that comprise basic amino acid residues, including residues of amino acids having two or more primary amine groups, such as lysine and ornithine, for example, polyamines that comprise from 2 to 10 lysine and/or ornithine amino acid residues (e.g., dilysine, trilysine, tetralysine, pentalysine, diornithine, triornithine, tetraornithine, pentaornithine, etc.).

Particular examples of polyamino compounds which may be used as the multifunctional compound include ethylenetriamine, diethylene triamine, hexamethylene triamine, di(heptamethylene) triamine, di(trimethylene) triamine, bis(hexamethylene) triamine, triethylene tetramine, tripropylene tetramine, tetraethylene pentamine, hexamethylene heptamine, pentaethylene hexamine, dimethyl octylamine, dimethyl decylamine, and JEFFAMINE polyetheramines available from Huntsman Corporation, chitosan and derivatives thereof, and poly(allyl amine), among others among others.

In some embodiments, the reactive compound having a plurality of second reactive moieties that comprise nucleophilic groups further comprises one or more radiopaque atoms, which may be selected, for example, from Br, I, Bi, Ba, Gd, Ta, Zn, W and Au.

Whether provided in the reactive multi-arm polymer containing a plurality of first reactive moieties that comprise an electrophilic group and/or in the reactive compound having a plurality of second reactive moieties that comprise nucleophilic groups, in some embodiments, the one or more radiopaque atoms are iodine atoms, which may be provided in a covalently attached iodinated moiety. In some of these embodiments, the iodinated moiety comprises an iodinated aromatic group. Examples of iodinated aromatic groups include iodine-substituted monocyclic aromatic groups and iodine-substituted multicyclic aromatic groups, such as iodinated phenyl groups, iodinated naphthyl groups, iodinated anthracenyl groups, iodinated phenanthrenyl groups, or iodinated tetracenyl groups. The iodinated aromatic groups may be substituted with one, two, three, four, five, six, or more iodine atoms. In some of these embodiments, the aromatic groups may be further substituted with one or more hydrophilic groups, for example, one, two, three, four, five, six or more hydrophilic groups. The hydrophilic groups may be hydroxyl-containing groups, which may be selected, for example, from hydroxyl groups and hydroxyalkyl groups (e.g., hydroxyalkyl groups containing one carbon, two carbons, three carbons, four carbons, etc.).

Specific examples of iodinated moieties include those that comprise one or more monocyclic or multicyclic aromatic structures, substituted with (a) one or more iodine groups (e.g., one two, three, four, five, six or more iodine atoms) and (b) optionally, one or more hydroxyl-containing groups independently selected from one or more hydroxyl groups and/or one or more C₁-C₄-hydroxyalkyl groups (e.g., C₁-C₄-monohydroxyalkyl groups, C₁-C₄-dihydroxyalkyl groups, C₁-C₄-trihydroxyalkyl groups, C₁-C₄-tetrahydroxyalkyl groups, etc.), among others, which C₁-C₄-hydroxyalkyl groups may be linked to the monocyclic or multicyclic aromatic structures directly or through any suitable linking moiety, which may be selected, for example, from ether groups, ester groups, amide groups, amine groups, or carbonate groups, among others.

Radiopaque crosslinked hydrogel particles may be provided by first combining (a) a reactive multi-arm polymer containing a plurality of first reactive moieties that comprise an electrophilic group, for example, as described hereinabove, and (b) a reactive compound having a plurality of second reactive moieties that comprise nucleophilic groups, for example, as described hereinabove, under conditions such that the electrophilic groups and the nucleophilic groups crosslink with one another. In certain embodiments, those conditions comprise an environment having a basic pH, for example, a pH ranging from about 9 to about 11.

The crosslinking reaction results in a radiopaque crosslinked hydrogel that may be reduced into hydrogel particles. For example, the radiopaque crosslinked hydrogel may be granulated into radiopaque crosslinked hydrogel particles of suitable size. Granulating may be by any suitable process, for instance, by processing the radiopaque crosslinked hydrogel in a homogenizer, grinding (including cryogrinding), crushing, milling, pounding, or the like. Sieving or other known techniques can be used to classify and fractionate the radiopaque crosslinked hydrogel particles. Radiopaque crosslinked hydrogel particles formed using the above and other techniques may varying widely in size, for example, having an average size ranging from 10 to 1500 um as previously noted.

Examples of radiopaque crosslinked hydrogel particles for use in the present disclosure also include polysaccharide particles that contain one or more crosslinked polysaccharides and one or more radiopaque atoms. Particular examples of crosslinked polysaccharide particles include hydrogel particles that contain crosslinked anionic polysaccharides, crosslinked cationic polysaccharides, and crosslinked neutral polysaccharides. Anionic polysaccharides include carboxylic-acid-containing polysaccharides that contain one or more uronic acid species, such as galacturonic acid, glucuronic acid and/or iduronic acid. Particular examples of carboxylic-acid-containing polysaccharides include pectin, agaropectin, carrageenan, gellan gum, gum arabic, guar gum, xanthan gum, alginic acid, hyaluronic acid, and carboxymethyl cellulose. In embodiments where the carboxylic-acid-containing polysaccharide is hyaluronic acid, the carboxylic-acid-containing polysaccharide may be non-animal stabilized hyaluronic acid. Another particular example of an anionic polysaccharide is agar, which is mixture of polysaccharides containing agarose, which is a neutral polysaccharide, and agaropectin, which is a charged sulfated polysaccharide. Other particular examples of anionic polysaccharides include carboxyalkyl celluloses such as carboxymethyl cellulose. Cationic polysaccharides include polysaccharides that contain positively charged functional groups such as amine functional groups, including primary amine groups, secondary amine groups, tertiary amine groups, and quaternary amine groups. Particular examples of cationic polysaccharides include chitosan and cationic starch. Neutral polysaccharides include cellulose derivatives including alkyl celluloses such as methyl cellulose and ethyl cellulose and hydroxyalkyl celluloses such as hydroxyethyl cellulose and hydroxypropyl cellulose. Neutral polysaccharides further include starches such as corn starch, potato starch, and tapioca starch.

The radiopaque atoms for use in the crosslinked polysaccharide particles may be selected, for example, from Br, I, Bi, Ba, Gd, Ta, Zn, W and Au. In some embodiments, where the one or more radiopaque atoms are iodine atoms, the one or more iodine atoms may be provided in a covalently attached iodinated moiety. In some of these embodiments, iodinated moiety comprises an iodinated aromatic group, examples of which are described above.

As previously noted, in various embodiments, the injectable hydrogel particle suspensions of the present disclosure contain one or more additional agents. Examples of such additional agents include therapeutic agents, imaging agents, colorants, tonicity adjusting agents, suspension agents and wetting agents.

Examples of therapeutic agents include antithrombotic agents, anticoagulant agents, antiplatelet agents, thrombolytic agents, anti-cancer drugs, antiproliferative agents, anti-inflammatory agents, hyperplasia inhibiting agents, anti-restenosis agents, steroids, anti-allergic agents, hemostatic agents, smooth muscle cell inhibitors, antibiotics, antimicrobials, anti-fungal agents, analgesics, anesthetics, immunosuppressants, growth factors, growth factor inhibitors, cell adhesion inhibitors, cell adhesion promoters, anti-angiogenic agents, cytotoxic agents, chemotherapeutic agents, checkpoint inhibitors, immune modulatory cytokines, T-cell agonists, and STING (stimulator of interferon genes) agonists, among others.

Examples of imaging agents include (a) fluorescent dyes such as fluorescein, indocyanine green, or fluorescent proteins (e.g. green, blue, cyan fluorescent proteins), (b) contrast agents for use in conjunction with magnetic resonance imaging (MRI), including contrast agents that contain elements that form paramagnetic ions, such as Gd(III), Mn(II), Fe(III) and compounds (including chelates) containing the same, such as gadolinium ion chelated with diethylenetriaminepentaacetic acid, (c) contrast agents for use in conjunction with ultrasound imaging, including organic and inorganic echogenic particles (i.e., particles that result in an increase in the reflected ultrasonic energy) or organic and inorganic echolucent particles (i.e., particles that result in a decrease in the reflected ultrasonic energy), (d) contrast agents for use in connection with near-infrared (NIR) imaging, which can be selected to impart near-infrared fluorescence to the hydrogel particle suspensions of the present disclosure, allowing for deep tissue imaging and device marking, for instance, NIR-sensitive nanoparticles such as gold nanoshells, carbon nanotubes (e.g., nanotubes derivatized with hydroxyl or carboxyl groups, for instance, partially oxidized carbon nanotubes), dye-containing nanoparticles, such as dye-doped nanofibers and dye-encapsulating nanoparticles, and semiconductor quantum dots, among others, and NIR-sensitive dyes such as cyanine dyes, squaraines, phthalocyanines, porphyrin derivatives and boron dipyrromethane (BODIPY) analogs, among others, (e) imageable radioisotopes including 99mTc, 201Th, 51Cr, 67Ga, 68Ga, 111In, 64Cu, 89Zr, 59Fe, 42K, 82Rb, 24Na, 45Ti, 44Sc, 51Cr and 177Lu, among others, and (f) radiocontrast agents (in addition to the radiopaque atoms in the crosslinked polysaccharide particles) such as metallic particles, for example, particles of tantalum, tungsten, rhenium, niobium, molybdenum, and their alloys, which metallic particles may be spherical or non-spherical. Additional examples of radiocontrast agents include non-ionic radiocontrast agents, such as iohexol, iodixanol, ioversol, iopamidol, ioxilan, or iopromide, ionic radiocontrast agents such as diatrizoate, iothalamate, metrizoate, or ioxaglate, and iodinated oils, including ethiodized poppyseed oil (available as Lipiodol®).

Examples of colorants include brilliant blue (e.g., Brilliant Blue FCF, also known as FD&C Blue 1), indigo carmine (also known as FD&C Blue 2), indigo carmine lake, FD&C Blue 1 lake, and methylene blue (also known as methylthioninium chloride), among others.

Examples of additional agents further include tonicity adjusting agents such as sugars (e.g., dextrose, lactose, etc.), polyhydric alcohols (e.g., glycerol, propylene glycol, mannitol, sorbitol, etc.) and inorganic salts (e.g., potassium chloride, sodium chloride, etc.), among others, suspension agents including various surfactants and wetting agents.

In various embodiments, the hydrogel particle suspensions of the present disclosure are visible under fluoroscopy. In various embodiments, the hydrogel particle suspensions have a radiopacity that is greater than 100 Hounsfield units (HU), beneficially anywhere ranging from 100 HU to 250 HU to 500 HU to 750 HU to 1000 IIU to 2000 HU or more (in other words, ranging between any two of the preceding numerical values). Such hydrogel particle suspensions can be used in a wide variety of biomedical applications, including implants, medical devices, and pharmaceutical compositions. The hydrogel particle suspensions of the present disclosure may be stored and transported in a sterile form. The hydrogel particle suspensions may be shipped, for example, in a syringe, catheter, vial, ampoule, or other container.

In various embodiments, kits are provided that include one or more delivery devices for delivering the hydrogel particle suspensions to a subject. Such kits may include one or more of the following: a syringe barrel, which may or may not contain a carrier fluid and/or radiopaque crosslinked hydrogel particles as described herein; a vial, which may or may not contain a carrier fluid and/or radiopaque crosslinked hydrogel particles as described herein; a needle; a flexible tube (e.g., a catheter adapted to fluidly connect the needle to the syringe); and an injectable liquid such as water for injection, normal saline or phosphate buffered saline. The carrier fluid will typically be provided in a syringe or a vial. Whether supplied in a syringe, vial, or other reservoir, the radiopaque crosslinked hydrogel particles may be provided in dry form (e.g., powder form) or in a form that is ready for injection (e.g., as a suspension of the radiopaque crosslinked hydrogel particles in the carrier fluid).

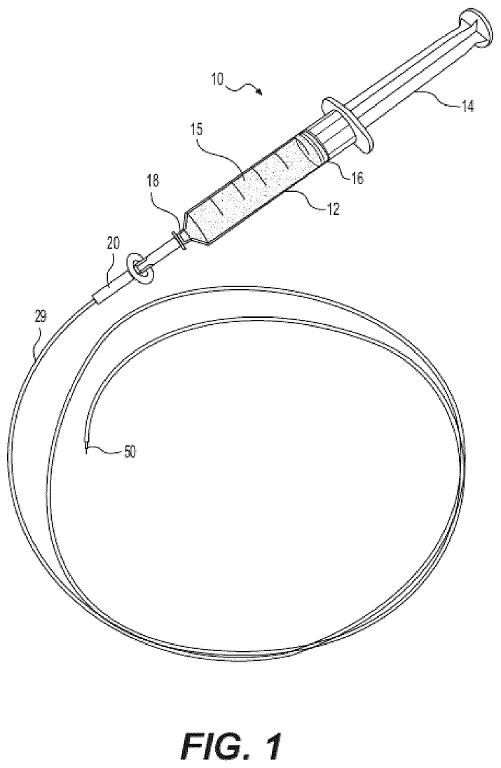

For example, FIG. 1 illustrates an exemplary syringe 10 providing a reservoir for a hydrogel particle suspension as described herein. The syringe 10 may comprise a barrel 12, a plunger 14, and one or more stoppers 16. The barrel 12 may include a Luer adapter (or other suitable adapter/connector), e.g., at the distal end 18 of the barrel 12, for attachment to an injection needle 50 via a flexible catheter 29. The proximal end of the catheter 29 may include a suitable connection 20 for receiving the barrel 12. In other examples, the barrel 12 may be directly coupled to an injection needle 50. The syringe barrel 12 may serve as a reservoir containing the hydrogel particle suspension 15 for injection through the needle 50.

The hydrogel particle suspensions described herein can be used for a number of purposes.

For example, hydrogel particle suspensions can be injected to provide spacing between tissues, hydrogel particle suspensions can be injected (e.g., in the form of blebs) to provide fiducial markers, hydrogel particle suspensions can be injected for tissue augmentation or regeneration, hydrogel particle suspensions can be injected as a filler or replacement for soft tissue, hydrogel particle suspensions can be injected to provide mechanical support for compromised tissue, hydrogel particle suspensions be injected as a scaffold, and/or hydrogel particle suspensions can be injected as a carrier of therapeutic agents in the treatment of diseases and cancers and the repair and regeneration of tissue, among other uses.

After administration, the hydrogel particle suspensions of the present disclosure can be imaged using a suitable imaging technique.

As seen from the above, the hydrogel particle suspensions of the present disclosure may be used in a variety of medical procedures, including the following, among others: a procedure to implant a fiducial marker comprising a radiopaque crosslinked hydrogel, a procedure to implant a tissue regeneration scaffold comprising a radiopaque crosslinked hydrogel, a procedure to implant a tissue support comprising a radiopaque crosslinked hydrogel, a procedure to implant a tissue bulking agent comprising a radiopaque crosslinked hydrogel, a procedure to implant a therapeutic-agent-containing depot comprising a radiopaque crosslinked hydrogel, a tissue augmentation procedure comprising implanting a radiopaque crosslinked hydrogel, a procedure to introduce a radiopaque crosslinked hydrogel between a first tissue and a second tissue to space the first tissue from the second tissue.

The hydrogel particle suspensions may be injected in conjunction with a variety of medical procedures including the following: injection between the prostate or vagina and the rectum for spacing in radiation therapy for rectal cancer, injection between the rectum and the prostate for spacing in radiation therapy for prostate cancer, subcutaneous injection for palliative treatment of prostate cancer, transurethral or submucosal injection for female stress urinary incontinence, intra- vesical injection for urinary incontinence, uterine cavity injection for Asherman's syndrome, submucosal injection for anal incontinence, percutaneous injection for heart failure, intra-myocardial injection for heart failure and dilated cardiomyopathy, trans-endocardial injection for myocardial infarction, intra-articular injection for osteoarthritis, spinal injection for spinal fusion, and spine, oral-maxillofacial and orthopedic trauma surgeries, spinal injection for posterolateral lumbar spinal fusion, intra-discal injection for degenerative disc disease, injection between pancreas and duodenum for imaging of pancreatic adenocarcinoma, resection bed injection for imaging of oropharyngeal cancer, injection around circumference of tumor bed for imaging of bladder carcinoma, submucosal injection for gastroenterological tumor and polyps, visceral pleura injection for lung biopsy, kidney injection for type 2 diabetes and chronic kidney disease, renal cortex injection for chronic kidney disease from congenital anomalies of kidney and urinary tract, intra-vitreal injection for neovascular age-related macular degeneration, intra-tympanic injection for sensorineural hearing loss, dermis injection for correction of wrinkles, creases and folds, signs of facial fat loss, volume loss, shallow to deep contour deficiencies, correction of depressed cutaneous scars, perioral rhytids, lip augmentation, facial lipoatrophy, stimulation of natural collagen production.

1. An injectable suspension comprising radiopaque crosslinked hydrogel particles in a carrier fluid that comprises one or more types of linear hydrophilic polymers.

2. The injectable suspension of claim 1, wherein the linear hydrophilic polymer is selected from a poly-2-oxazoline, a polyvinylpyrrolidone, and a polyacrylamide.

3. The injectable suspension of claim 1, wherein the linear hydrophilic polymer has a weight average molecular weight (M_{w}) ranging from 1 kDa to 60 kDa.

4. The injectable suspension of claim 1, wherein the carrier fluid contains between 1 wt % and 35 wt % of the one or more types of linear hydrophilic polymers.

5. The injectable suspension of claim 1, wherein the radiopaque crosslinked hydrogel particles comprise one or more radiopaque atoms selected from Br, I, Bi, Ba, Gd, Ta, Zn, W and Au.

6. The injectable suspension of claim 1, wherein the radiopaque crosslinked hydrogel particles comprise covalently attached iodine atoms.

7. The injectable suspension of claim 1, wherein the radiopaque crosslinked hydrogel particles comprise a crosslinked polymer that comprises one or more of the following:
a poly(alkylene oxide) chain, a poly(N-vinyl pyrrolidone) chain, a poly(2-oxazoline) chain, a polyacrylamide chain, a polysaccharide chain, a polypeptide chain, a polyvinyl alcohol chain, and a poly(2-hydroxyethyl methacrylate) chain.

8. The injectable suspension of claim 1, wherein the radiopaque crosslinked hydrogel particles comprise a crosslinked polymer having a core region and a plurality of polymer arms linked to the core region, the polymer arms each comprising a hydrophilic polymer chain.

9. The injectable suspension of claim 8, wherein a portion of the polymer arms comprise a radiopaque-atom-containing moiety at the end.

10. The injectable suspension of claim 1, wherein the radiopaque crosslinked hydrogel particles comprise a crosslinked reaction product of (a) a reactive multi-arm polymer containing a plurality of first reactive moieties that comprise an electrophilic group and (b) a reactive compound having a plurality of second reactive moieties that comprise nucleophilic groups.

11. The injectable suspension of claim 1, wherein the injectable suspension contains between 1 wt % and 25 wt % (dry weight) radiopaque crosslinked hydrogel particles relative to the total weight of the suspension.

12. The injectable suspension of claim 1, wherein the radiopaque crosslinked hydrogel particles range 10 to 1500 microns in in longest dimension.

13. The injectable suspension of claim 1, wherein the carrier fluid contains an acidic buffer and has a pH ranging from 3 to 6.5.

14. The injectable suspension of claim 1, wherein the carrier fluid further comprises a therapeutic agent.

15. The injectable suspension of claim 1, wherein the injectable suspension has a radiopacity that is greater than 100 Hounsfield units (HU).

16. The injectable suspension of claim 1, preloaded in a syringe.

17. A kit comprising (a) a syringe preloaded with an injectable suspension comprising radiopaque crosslinked hydrogel particles in a carrier fluid that comprises one or more types of linear hydrophilic polymers and (b) a delivery device.

18. The kit of claim 17, wherein the delivery device comprises a catheter and/or a needle.

19. A method of treatment comprising injecting into a subject an injectable suspension comprising radiopaque crosslinked hydrogel particles in a carrier fluid that comprises one or more types of linear hydrophilic polymers.

20. The method of claim 19, wherein the hydrogel particle suspension is injected to provide spacing between tissues, injected to provide fiducial markers, injected for tissue augmentation or regeneration, injected as a filler or replacement for soft tissue, injected to provide mechanical support for compromised tissue, injected as a scaffold, and/or injected as a carrier for one or more therapeutic agents.
